(19) United States
(12) Patent Application Publication
(10) Pub. No.: US 2024/0057984 A1
(43) Pub. Date: Feb. 22, 2024

(54) COLLECTION DEVICE

(71) Applicant: H & B Electronic GmbH & Co. KG, Deckenpfronn (DE)

(72) Inventors: Tobias MORLOK, Mötzingen (DE); Jan BAYERBACH, Calw (DE); Jochen HEIMSCH, Fellbach (DE); Nico REMY, Tübingen (DE)

(73) Assignee: H & B Electronic GmbH & Co. KG, Deckenpfronn (DE)

(21) Appl. No.: 18/226,540

(22) Filed: Jul. 26, 2023

(30) Foreign Application Priority Data
Aug. 19, 2022 (DE) 102022121041.3

(51) Int. Cl. A61B 10/02

(52) U.S. Cl. CPC A61B 10/025; A61B 10/0283

(57) ABSTRACT

A collection device for a tissue removal device has a flow connection on the shaver side, which can be fluidically connected to a tissue discharge of the tissue removal device, a flow connection on the vacuum side, which can be fluidically connected to a vacuum device, a connection path which can be formed between the flow connection on the shaver side and the flow connection on the vacuum side and via which a fluid flow can be formed from the flow connection on the shaver side to the flow connection on the vacuum side, and a collection container which can be attached in the connection path for receiving tissue material from the fluid flow. A bypass path separate from the collection container can be formed between the flow connection on the shaver side and the flow connection on the vacuum side. The flow connection on the shaver side and the flow connection on the vacuum side are provided on a connection housing on which the collection container is held in a removable manner.

The invention relates to a collection device for a tissue removal device, such as in particular a shaver, according to the preamble of claim 1. The collection device serves to retain and collect tissue material removed from a patient by means of the shaver, such as in particular autologous cartilage tissue of a joint or bone material. The collected tissue material is intended to be used in this case, when mixed with a medium, such as in particular a hydrogel or blood serum, for the preparation and production of an in particular autologous applicator mass. For this purpose, the collection device has a flow connection on the shaver side, which can be fluidically connected to a tissue discharge of the tissue removal device, a flow connection on the vacuum side, which can be fluidically connected with a vacuum device, and a connection path that can be formed between the flow connection on the shaver side and the flow connection on the vacuum side. A fluid flow can be formed via this connection path from the flow connection on the shaver side to the flow connection on the vacuum side, in which the removed tissue material is transported away from the tissue removal device. In addition, a collection container is provided which can be arranged in the connection path and which serves to receive tissue material separated from the fluid flow. A bypass path separate from the collection container can be formed between the flow connection on the shaver side and the flow connection on the vacuum side. As a result, the fluid flow between the two flow connections can also be formed independently of the collection container.

US 2021/0251647 A1 discloses a device for collecting tissue which has a flow connection on the shaver side and a flow connection on the vacuum side. In between, the collection device forms a connection path in the form of a multi-part, sleeve-shaped collection container, in which a filter part with a plurality of filter elements with different permeability is accommodated. Two housing parts of the collection container can be separated from one another in order to be able to access the tissue material held back by the filter part.

A disadvantage of the known collection device is that it has to be dismantled in a relatively complicated manner in order to remove the tissue material and the relevant shaver cannot be operated during such a tissue removal.

CN107744404A describes a patellar tissue collection device having a collection container between a shaver and a vacuum device. A bypass line is also provided so that the container can be emptied or replaced during use. To activate the bypass, a closing valve is provided at the inlet and at the outlet of the collection container and in the bypass line. The valves can be controlled depending on a measuring sensor that is provided in a further parallel measuring line.

The object of the invention is to avoid the disadvantages mentioned in a generic collection device and to enable easier and faster removal of the retained tissue and more convenient operation of the associated tissue removal device.

This object is achieved by a collection device having the features of claim 1. In this case, the flow connection on the shaver side and the flow connection on the vacuum side are arranged on a connection housing on which the collection container is held in a removable manner. This makes it possible to separate the collection container for removing the retained tissue material from the connection housing and thus from the rest of the tissue collection arrangement, which consists of the tissue removal device, the connection housing, the vacuum device and the flow connections between them. This enables a particularly simple and convenient removal and preparation of the retained tissue material. In this way, it is possible to operate the tissue removal device even while retained tissue material is being removed from the collection container, since the tissue material removed by the shaver can continue to be transported away via a fluid flow.

Advantageously, a switching device is provided, via which the flow connection on the shaver side can be connected to the flow connection on the vacuum side either via the connection path or the bypass path. In this way, the collection device can be switched between a collection mode and a pure suction mode, in which the tissue removed by the shaver can be transported away without being collected. This in turn makes it possible to empty the collection container while the shaver continues to be operated in parallel in pure suction mode.

The switching device is preferably formed by a two-way valve in order to enable rapid switching between collection mode and suction mode without any significant interruption.

In addition, it is advantageous if the switching device can be switched via a rotary member in order to enable particularly comfortable operation, in particular with one hand. As an alternative to the rotary member, the switching device can also have a sliding member or any other known handle that is suitable for switching a two-way valve.

In this case, it is particularly advantageous if the bypass path is formed within the connection housing, so that the rest of the tissue collection arrangement can also be operated in pure suction operation without the collection container.

The collection container preferably forms a flow portion of the connection path between an inlet opening and an outlet opening, in which a collection receptacle delimited by a sieve device is provided. As a result, the connection path can be built up via the collection container during collection operation and the tissue can be separated from the connection path in a simple manner and over a relatively large filter region.

The sieve device is preferably formed by a flat sieve surface mounted in front of the outlet opening in the direction of flow. The planar design of the sieve surface makes it easier to remove tissue particles adhering during the collection operation or an adhering filter cake.

Furthermore, the collection receptacle can advantageously be observed via a transparent region of the collection container, such as through a window or a transparent wall of the collection container. In this way, it can be determined in a simple manner in the course of the collection operation whether a sufficient quantity of the respective tissue material has been collected. In addition, a level scale can be provided on the transparent region, which makes it easier to estimate the amount of material collected.

Furthermore, it is advantageous if a closing element is held on the removable collection container, which can be adjusted between a first open position, in which the inlet opening of the collection receptacle is released, and a closed position, in which the inlet opening is closed. As a result, the collection container can be opened via the closing element on the one hand in order to release the connection path for collection operation in the position in which it is attached to the connection housing. On the other hand, the collection container can be closed via the closing element in order to avoid an unintentional escape of retained tissue material or its contamination by entering impurities when the collection container is separated from the connection housing.

It is advantageous if the closing element can be brought automatically into the first open position when the collection container is attached to the connection housing, in order to ensure that the connection path is released when the collection container is attached or during collection operation.

It is also advantageous if the closing element can be brought into the closed position automatically when it is removed from the collection container, in order to prevent the tissue material contained in it from escaping unintentionally during separation and when the collection container is separated from the connection housing.

In this case, the closing element advantageously has an actuating element, via which the closing element can be adjusted into the first open position when the collection container is attached, by interacting with the connection housing. For example, the actuating element is formed by a contact cam that can be placed against the connection housing and thus displaced relative to the collection housing. As a result, the automatic displacement of the closing element into the first open position can be implemented with particularly simple means and low production costs.

In addition, it is advantageous if the closing element is biased into the closed position. As a result, the collection container can be securely closed both during removal from the connection housing and when completely separated from it, in order to prevent accidental leakage and/or contamination of the collected tissue material.

Advantageously, the collection receptacle can also be emptied by means of a piston in a second open position of the closing element. This enables a particularly comfortable and complete removal of the collected tissue material from the collection container.

It is advantageous if the collection container has a container opening that can be closed by the closing element and the piston is held on a side of the collection receptacle facing away from the closing element and can be displaced along the collection receptacle in the direction of the container opening. In this way, in the second open position of the closing element, the collection receptacle can be conveniently and completely emptied by means of the piston.

Furthermore, it is advantageous if the piston can be displaced along an inner surface of the sieve device, which means that retained tissue particles adhering to the sieve device can also be removed in a convenient manner, in order to be able to provide the collected tissue material as completely as possible for processing or later use.

It should be noted that all features of the subject matter according to the invention described above can be swapped or combined with one another, provided that a swap or a combination thereof is not ruled out for technical reasons.

An exemplary embodiment of the invention is shown in the figures. In particular:

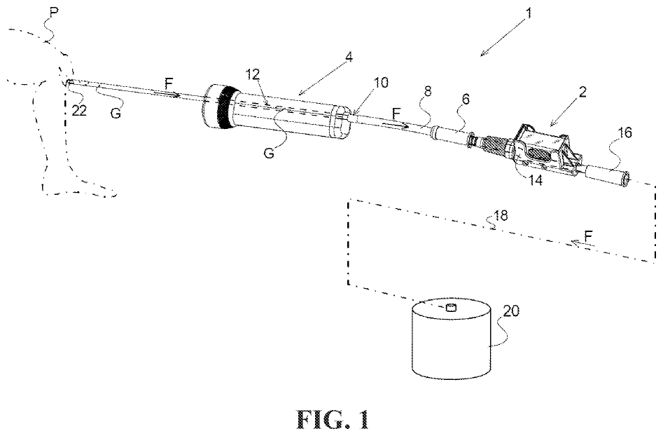

FIG. 1 shows a tissue collection arrangement with a collection device according to the invention,

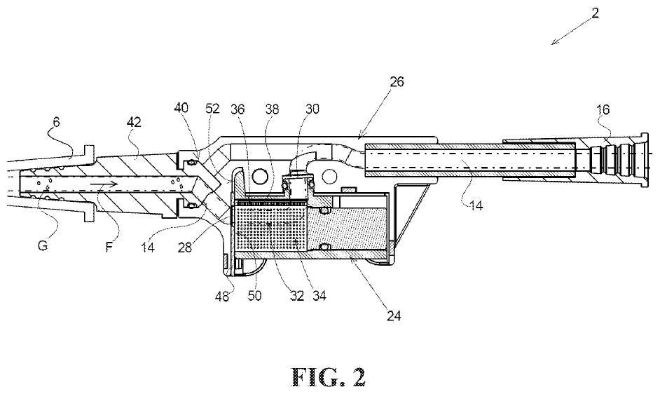

FIG. 2 shows a longitudinal section through the collection device according to FIG. 1 with an formed connection path,

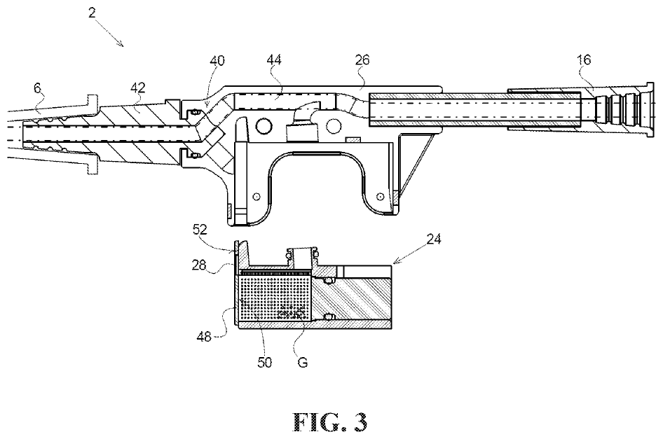

FIG. 3 shows a longitudinal section through the collection device according to FIG. 1 with a formed bypass path,

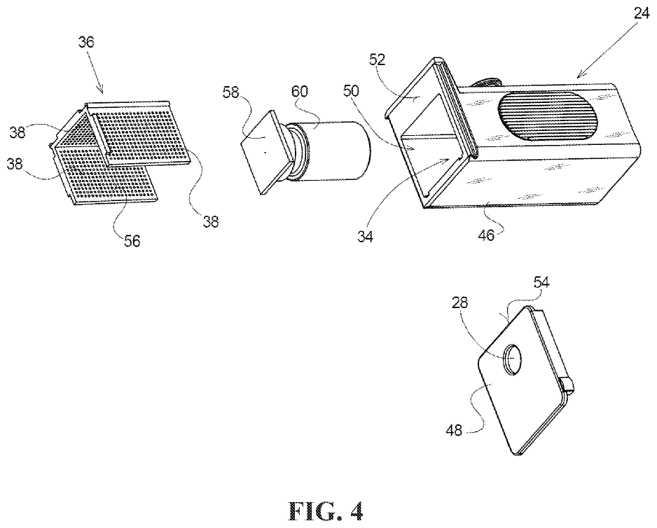

FIG. 4 shows an exploded view of a collection container of the collection device according to FIG. 1.

FIG. 1 shows a tissue collection arrangement 1 with a collection device 2, a tissue removal device 4 and a vacuum device 20. The tissue removal device 4 is designed in the form of a shaver, by means of which tissue material G, such as in particular cartilage tissue or bone material, can be removed from a patient P. The removal takes place, for example, on a knee, a shoulder joint or on another bone. The tissue material G obtained in this way is used for the later production of an in particular autologous applicator mass, which can be used in the treatment of a lesion or in the build-up of tissue or bone on a defect in the patient P.

For collecting the tissue material G, the collection device 2 has a flow connection 6 on the shaver side, which is fluidically connected via a connection line 8 to a delivery connection 10 of a tissue discharge 12 of the tissue removal device 4. In addition, the collection device 2 has a vacuum-side flow connection 16 which can be connected to the shaver-side flow connection 6 via a connection path 14 and which is fluidically connected to a vacuum device 20 via a connection line 18.

As soon as a vacuum is applied to the connection line 18 by means of the vacuum device 20, a fluid flow F is thereby generated from the tissue removal device 4 via the collection device 2 to the vacuum device 20. The tissue material G removed by a tool 22 can be transported in the fluid flow F to the discharge connection 10 via the tissue discharge 12, via which it is passed on to the collection device 2.

As can be seen in particular from FIG. 2, the collection device 2 has a collection container 24 which is held on a connection housing 26. The collection container 24 forms a flow portion 32 between an inlet opening 28 and an outlet opening 30, which portion runs through a collection receptacle 34. The collection receptacle 34 is partially delimited by a sieve device 36 which has at least one planar sieve surface 38 in front of the outlet opening 30 in the direction of flow of the fluid flow F.

As can also be seen from FIG. 2, the connection path 14 runs from the flow connection 6 on the shaver side via the flow portion 32 to the flow connection 16 on the vacuum side. In this way, the tissue material G transported with the fluid flow can be separated by the sieve device 36 and collected in the collection receptacle 34.

Between the flow connection 6 on the shaver side and the collection container 24 there is also a two-way valve which acts as a switching device 40 and can be switched between a connecting position and a bypass position. For this purpose, the switching device 40 has an actuator 42 which, as shown, can be designed as a rotary member. As an alternative, the actuator 42 can also be designed as a sliding member or in any other known form suitable for switching over a two-way valve (not shown).

In any case, in the connection position of the two-way valve 40 shown in FIG. 2, the flow connection 6 on the shaver side is connected to the flow connection 16 on the vacuum side via the connection path 14, so that the tissue collection arrangement 1 can be used with the collection device 2, the tissue removal device 4 and the vacuum device 20 in a collection operation.

By rotating the actuator 42, the two-way valve 40 can be brought into the bypass position according to FIG. 3. In this position, the flow connection 6 on the shaver side is now connected to the flow connection 16 on the vacuum side via a bypass path 44. In this case, the bypass path 44 is integrated into the connection housing 26 separately from the collection container 24. As a result, the collection device 2 can be used together with the tissue removal device 4 and the vacuum device 20 in a pure suction operation, which takes place independently of the collection container 24. As a result, the collection container 24 can be removed from the connection housing 26 also during operation of the tissue removal device 4, as shown, in order, for example, to remove the tissue material G previously collected therein. In order to be able to maintain the suction capacity also via the bypass path 44, a closing element can be provided on the outlet opening 30 on the side of the connection housing 26 (not shown).

An at least partially transparent region 46 is provided on the collection container 24 according to FIG. 4, through which the collection receptacle 34 can be viewed. The transparent region 46 can, as shown, be formed by making the collection container 24 itself completely or partially transparent. As an alternative, the transparent region 46 can also be formed by a window which is provided in the otherwise non-transparent collection container 24 (not shown). In this way it can be checked whether a sufficient amount of the removed tissue material G has been collected. For a better assessment of the amount of tissue material G retained in the collection receptacle 34, a measuring scale (not shown) can be provided on the transparent region or in the region that can be seen from it.

As can also be seen from FIG. 4, the inlet opening 28 of the collection receptacle 34 is integrated, for example, in a closing element 48 which can be adjusted at a container opening 50 of the collection container 24 between a first and second open position and a closed position. In the closed position, the inlet opening 28 is at the level of a closed outer surface 52 of the collection container 24, so that the collection receptacle 34 is closed by the rest of the closing element 48, as shown in FIG. 3.

In the first open position, on the other hand, the inlet opening 28 is at the level of the collection receptacle 34, so that the connection path 14 can be formed via this, as shown in FIG. 2.

The closing element 48 is designed in such a way that it is automatically brought into the first open position when the collection container 24 is attached to the connection housing 26. For this purpose, the closing element 48 has an actuating element 54 which, as shown, is formed, for example, by an edge of the closing element 48 or by an additionally formed contact cam (not shown) and which comes into contact with the connection housing 26 when the collection container 24 is attached, and thereby the closing element 48 is shifted from the closed position to the open position.

In addition, the closing element 48 can be automatically displaced into the closed position when it is removed from the collection container. For this purpose, a spring device (not shown), for example, can be provided between the closing element 48 and the rest of the collection container 24, which device biases the closing element 48 into the closed position.

As can also be seen from FIG. 4, the sieve device 36 has, for example, three planar sieve surfaces 38 along the respective inner surface 56 of which a scraper 58 can be displaced, which scraper is held on a piston 60. The piston 60 is in turn held displaceably at an end of the collection receptacle 34 which is facing away from the inlet opening 28 or from the closing element 48, as can be seen from FIGS. 2 and 3. As a result, the piston 60 can be displaced along the collection receptacle 34 in the direction of the container opening 50. In this way, in the second open position of the closing element 48, in which it releases the container opening 50 essentially completely, the collected tissue material G can be discharged completely from the collection receptacle 34. The scraper 58 carried on the piston 60 also loosens tissue particles adhering to the sieve surfaces 38 and likewise discharges them via the container opening 50 in order to ensure that the collection receptacle 34 is essentially completely emptied.

It should be noted that all elements and features of the various embodiments of the subject matter according to the invention described above can be swapped or combined with one another, provided that a swap or a combination thereof is not ruled out for technical reasons.

1. A collection device for a tissue removal device, comprising:
a flow connection on a shaver side, which can be fluidically connected to a tissue discharge of the tissue removal device,
a flow connection on a vacuum side, which can be fluidically connected to a vacuum device,
a connection path which can be formed between the flow connection on the shaver side and the flow connection on the vacuum side and via which a fluid flow can be formed from the flow connection on the shaver side to the flow connection on the vacuum side; and
a collection container which can be attached in the connection path for receiving tissue material from the fluid flow,
wherein a bypass path separate from the collection container can be formed between the flow connection on the shaver side and the flow connection on the vacuum side, and
wherein the flow connection on the shaver side and the flow connection on the vacuum side are provided on a connection housing on which the collection container is held in a removable manner.

2. The collection device according to claim 1, wherein a switching device is provided, via which the flow connection on the shaver side can be selectively connected to the flow connection on the vacuum side, via the connection path or the bypass path.

3. The collection device according to claim 2, wherein the switching device is formed by a two-way valve.

4. The collection device according to claim 2, wherein the switching device can be switched via a rotary member.

5. The collection device according to claim 1, wherein the bypass path is formed within the connection housing.

6. The collection device according to claim 1, wherein the collection container forms a flow portion of the connection path between an inlet opening and an outlet opening, wherein a collection receptacle delimited by a sieve device is provided in the flow portion.

7. The collection device according to claim 6, wherein the sieve device is formed by a planar sieve surface, which is attached upstream of the outlet opening in the direction of flow.

8. The collection device according to claim 6, wherein the collection receptacle can be observed through a transparent region of the collection container.

9. The collection device according to claim 6, wherein a closing element is held on the removable collection container and can be adjusted between a first open position in which the inlet opening of the collection receptacle is released, and a closed position, in which the inlet opening is closed.

10. The collection device according to claim 9, wherein the closing element can be brought automatically into the first open position when the collection container is attached to the connection housing.

11. The collection device according to claim 9, wherein the closing element can be brought into the closed position automatically when it is removed from the collection container.

12. The collection device according to claim 9, wherein the closing element has an actuating element via which the closing element is adjustable into the first open position when the collection container is attached, by interacting with the connection housing.

13. The collection device according to claim 9, wherein the closing element is biased into the closed position.

14. The collection device according to claim 6, wherein the collection receptacle can be emptied by means of a piston.

15. The collection device according to claim 14, wherein the collection container has a container opening which can be closed by the closing element and which is released in a second open position of the closing element, and the piston is held on one side of the collection receptacle facing away from the closing element and can be displaced along the collection receptacle in the direction of the container opening.

16. The collection device according to claim 14, wherein the piston is displaceable along an inner surface of the sieve device.

17. The collection device according to claim 3, wherein the switching device can be switched via a rotary member.

18. The collection device according to claim 2, wherein the bypass path is formed within the connection housing.

19. The collection device according to claim 3, wherein the bypass path is formed within the connection housing.

20. The collection device according to claim 4, wherein the bypass path is formed within the connection housing.
